(19) United States
(12) Patent Application Publication
(10) Pub. No.: US 2025/0256077 A1
(43) Pub. Date: Aug. 14, 2025

(54) FOAM-TYPE MATERIAL APPLICATOR

(71) Applicant: Young CHOI, Seoul (KR)

(72) Inventor: Young CHOI, Seoul (KR)

(21) Appl. No.: 18/856,814

(22) PCT Filed: Nov. 30, 2022
(86) PCT No.: PCT/KR2022/019263

§ 371 Date: Oct. 14, 2024

(30) Foreign Application Priority Data
Apr. 18, 2022 (KR) 10-2022-0047679

(51) Int. Cl. A61M 35/00

(52) U.S. Cl. CPC A61M 35/003; A61M 2210/04

(57) ABSTRACT

The present invention relates to a foam-type material applicator and, in particular, to a foam-type material applicator comprising an application body in which a piston provided thereinside advances by a certain distance when a button is pressed, and a temperature-sensitive foam-type material accommodated thereinside is discharged in a fixed amount through a nozzle to the outside near an application area such as the scalp, wherein the application body is configured such that the piston remains in an advanced state when the button is released such that a certain amount of the foam-type material is discharged whenever the button is pressed.

### TECHNICAL FIELD

The present disclosure relates to a foam-type material applicator and, more particularly, to a foam-type material applicator that discharges a certain amount of foam-type material so that the foam-type material can be effectively applied to the skin, including the scalp.

### BACKGROUND ART

In recent years, as the age at which hair loss begins decreases, the importance of early hair loss management is increasing. Early hair loss can be prevented by changing lifestyle habits and visiting a scalp clinic for proper care, but severe hair loss needs to be treated with medication or treatment. As the importance of consistent hair loss management is emphasized, topical hair loss treatments that can be easily used at home at a relatively low cost are attracting the attention of consumers.

The topical hair loss treatments contain minoxidil as the main ingredient, which helps hair growth by dilating blood vessels in the scalp. Initially developed as a liquid type, a foam version of topical minoxidil that solved problems such as dripping when applied was later released. Due to the nature of the foam-type hair loss treatment, the minoxidil foam does not flow out, and thus the foam can be applied in an appropriate amount only to the affected area of the scalp, and has the advantage of being absorbed into the scalp and drying quickly.

When using a typical foam-type hair loss treatment, a user should squeeze a certain amount of foam into the cap of the product, then apply the foam to the scalp with his/her fingers. However, because the fingerbreadth is large, while trying to apply the foam to the scalp, a significant amount is unwantedly applied to the hair around the hair loss area, which reduces efficiency and results in the failure to meet expectations for hair growth effects. It especially gets worse when the user applies the foam with his/her fingers to areas of the scalp that are hard to see, such as the crown or the back of the head. Moreover, if the foam turns into liquid due to the body temperature of the fingers, it becomes more difficult to apply to the scalp.

Eventually, a larger amount is used to meet hair growth expectations, and it becomes impossible to confirm whether the correct amount of minoxidil (1 g per time) was used. When more than the recommended amount of minoxidil is used, side effects such as headaches and eye tenderness may occur due to excessive vasodilation.

### DISCLOSURE

### Technical Problem

The present disclosure is intended to solve the above problems occurring in the related art. An objective of the present disclosure is to provide a foam-type material applicator that discharges a fixed amount of temperature-sensitive foam-type material near an application area, such as the scalp.

### Technical Solution

In order to achieve the above-mentioned objectives, there is provided a foam-type material applicator including: an applicator body in which a piston provided thereinside advances by a certain distance when a button is pressed so that a foam-type material accommodated thereinside is discharged outside through a nozzle, wherein the applicator body may be configured such that the piston remains in an advanced state when the button is released so that a certain amount of the foam-type material may be discharged whenever the button is pressed.

In addition, the piston may be provided with a movement guide hole recessed from a top thereof downward, and the applicator body may further include a pad member provided at a certain position in the movement guide hole, configured to contact a wall of the movement guide hole and advance the piston a certain distance when the button is pressed, and spaced apart from the wall of the movement guide hole when the button is released.

In addition, the pad member may include: a pad support (410) fixed at a certain position in the movement guide hole (210); and a pad movably coupled to the pad support, and configured to move downwardly outward when the button is pressed and push the wall of the movement guide hole to move the piston a certain distance.

In addition, the button may include a pressing column provided in the movement guide hole to face the pad member so as to press the pad member, the applicator body may include a guide container in which a guide column is provided that surrounds the pressing column within the movement guide hole and provides a movement path for the pressing column, and in which the piston is provided thereinside, and the pad support may be fixed to a bottom of the guide column.

In addition, the applicator body may include an accommodation container configured to accommodate the foam-type material thereinside and keep the foam-type material from changing phase, and the nozzle may be detachably coupled to a bottom of the accommodation container.

### Advantageous Effects

According to the present disclosure, a foam-type material can be discharged close to an application area such as the scalp. Thus, compared to a conventional application method using fingers, the temperature-sensitive foam-type material can be efficiently applied to the application area, which is the target area, without phase change.

Particularly, when applying to hard-to-see areas such as the top of the head, by using an applicator, unlike the conventional method of applying based on the feel of user's fingertips, a user can apply the foam more easily and conveniently by simply placing a nozzle close to the application area and pressing a button.

Furthermore, compared to the conventional application method using fingers, the applicator discharges a certain amount of foam over a small area, allowing a user to spread the foam evenly without clumping over a wider area, maximizing efficiency.

As a result, the amount applied to the target area, the scalp, is increased compared to the conventional case, thereby meeting the expected effect on hair growth and reducing product abuse/overuse.

### DESCRIPTION OF DRAWINGS

FIG. 1 is a perspective view showing a foam-type material applicator of the present disclosure.

FIG. 2 is an exploded perspective view showing the foam-type material applicator of the present disclosure.

FIG. 3 is a cut-off exploded perspective view showing the foam-type material applicator of the present disclosure.

FIG. 4 is a cross-sectional view showing the foam-type material applicator of the present disclosure.

FIG. 5 is a cross-sectional view showing the structure of an accommodation container applied to the foam-type material applicator of the present disclosure.

FIG. 6 is an exploded perspective view showing a pad member applied to the foam-type material applicator of the present disclosure.

FIG. 7 is a cross-sectional view showing a state in which a pad support and a guide column applied to the foam-type material applicator of the present disclosure are combined.

FIG. 8 is a cross-sectional view showing a piston advancing a certain distance when a button applied to the foam-type material applicator of the present disclosure is pressed.

FIG. 9 is a cross-sectional view showing that the piston maintains an advanced state when the button applied to the foam-type material applicator of the present disclosure is released.

FIG. 10 is a view showing a comparative example of applying a foam-type material using fingers and the present disclosure.

FIG. 11 is an exemplary view showing the use of the foam-type material applicator of the present disclosure.

### MODE FOR INVENTION

In the present disclosure, proposed is a foam-type material applicator including an applicator body in which a piston provided thereinside advances by a certain distance when a button is pressed, and a temperature-sensitive foam-type material accommodated thereinside is discharged in a fixed amount through a nozzle to the outside near an application area such as the scalp, wherein the applicator body is configured such that the piston remains in an advanced state when the button is released such that a certain amount of the foam-type material is discharged whenever the button is pressed.

The scope of the present disclosure is not limited to the embodiments described below, and various modifications may be made by a person having ordinary knowledge in the relevant technical field without departing from the technical spirit of the present disclosure.

Hereinafter, a foam-type material applicator of the present disclosure will be described in detail with reference to the attached drawings 1 to 11.

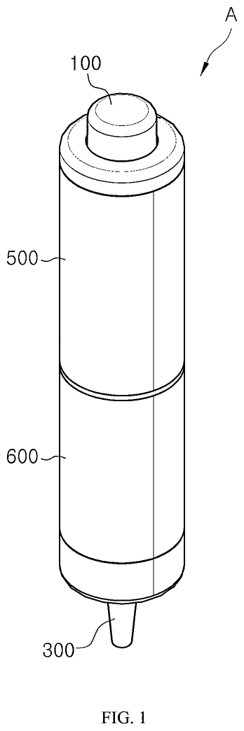

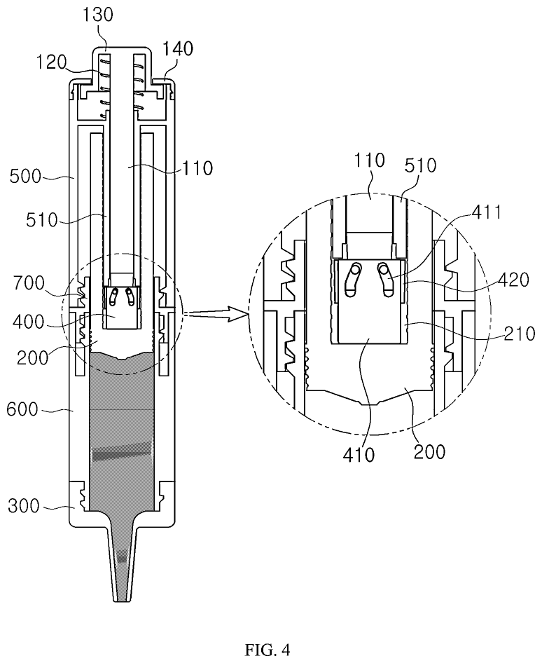

As shown in FIGS. 1 to 4, the foam-type material applicator of the present disclosure includes an applicator body A that discharges a certain amount of a foam-type material contained thereinside to the outside. In this case, the foam-type material may be substances with various ingredients such as hair loss agents and nutrients that form a foam, and may also be temperature-sensitive substances that change phase from a solid to a liquid when heat is applied.

In the present disclosure, the applicator body A includes a button 100, a piston 200, and a nozzle 300. In the following description of the applicator body A, the side provided with the nozzle 300 is defined as the downward direction, and the opposite side is defined as the upward direction. The applicator body A is configured such that when the button 100 is pressed, the piston 200 provided inside the applicator body A advances a certain distance and pressurizes the foam-type material contained therein so that the foam-type material is discharged to the outside through the nozzle 300. At this time, the certain distance preferably means a distance equal to or shorter than half the maximum distance that the piston 200 can move within the applicator body A. That is, the applicator body A may be configured so that all of the foam-type material contained therein may be discharged to the outside only when the button 100 is pressed at least twice.

In addition, the applicator body A is configured to maintain the piston 200 in an advanced state when the button 100 pressed to discharge the foam-type material to the outside is released. As an example, when the button 100 is pressed once for the first time, the piston 200 moves 0.25 mm toward the nozzle 300 and a certain amount of foam-type material is discharged to the outside. At this time, when the button 100 is released, the piston 200 remains moved by 0.25 mm toward the nozzle 300. Afterwards, when the button 100 is pressed once again, the piston 200 moves 0.25 mm from the point where the piston 200 is first advanced toward the nozzle 300, and a certain amount of foam-type material is ejected. That is, when the button 100 is pressed twice and then released, the piston 200 is positioned at a point moved by 0.5 mm from the initial position thereof toward the nozzle 300.

As such, since the piston 200 does not return to the initial position thereof when the button 100 is released, the piston 200 gradually advances each time the button 100 is pressed, thereby ejecting a certain amount of foam-type material each time. Thus, it is possible to spread and apply a certain amount of foam-type material evenly to various targeted scalp areas, thereby reducing side effects such as pain caused when applying a large amount of foam to one area.

As a specific example, the applicator body A includes the button 100, the piston 200, and the nozzle 300 as shown in FIGS. 1 to 4, and may further include a pad member 400, a guide container 500, an accommodation container 600, and a joint 700.

First, the accommodation container 600, the nozzle 300, and the joint 700 will be described. The accommodation container 600 is open at both ends and is formed in the form of an empty cylinder, and the foam-type material may be accommodated therein. The nozzle 300 may be coupled to the lower end of the accommodation container 600, and the joint 700 for connection to the guide container 500 may be coupled to the upper end of the accommodation container 600.

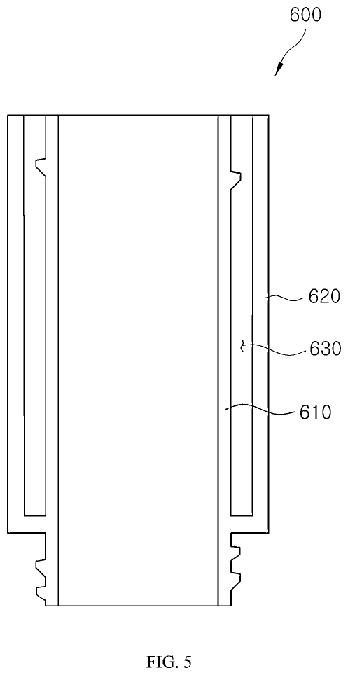

The accommodation container 600 may be configured to maintain the foam-type material contained therein from undergoing phase change. As an example, the wall of the accommodation container 600 may have a double structure. That is, as shown in FIG. 5, the accommodation container 600 may consist of an inner container 610 in which the foam-type material contained therein is accommodated, and an outer container 620 surrounding the inner container 610 and forming a filling space 630 between the inner container 610 and the outer container 620.

To be specific, the inner container 610 may be formed to have a longer length than the outer container 620, so that the lower end of the inner container 610 protrudes downward than the outer container 620, and the nozzle 300 may be coupled to the lower end of the inner container 610. The lower end of the outer container 620 is connected to the inner container 610 to form a structure in which the lower side of the filling space 630 is blocked. Accordingly, a material that prevents the phase change of the foam-type material may be injected into the filling space 630 through the upper side of the filling space 630, which can be opened by separating the joint 700 coupled to the top of the accommodation container 600. At this time, the material that prevents the phase change of the foam-type material may be air, a cooling material, etc.

In this case, after the material that prevents phase change of the foam-type material is injected into the filling space 630, the top of the accommodation container 600 and the joint 700 coupled thereto may be separately coupled to enable additional injection. The top of the accommodation container 600 and the joint 700 coupled thereto may be bonded to prevent additional injection.

Meanwhile, the nozzle 300 may include a coupling portion coupled to the lower end of the inner container 610 and a discharge portion extending downward from the coupling portion, and the coupling portion may be detachably coupled to the lower end of the accommodation container 600. The joint 700 connects the accommodation container 600 and the guide container 500 to each other and serves to seal the filling space 630. As an example, the joint 700 may include: a sealing portion that forms a ring shape and covers the top of the accommodation container 600 to seal the filling space 630; a first joint portion extending downward from the sealing portion and screwed to the inner container 610; and a second joint portion extending upward from the sealing portion and screwed to the guide container 500. Due to the joint 700, the accommodation container 600 may be separated from the guide container 500.

Thus, if cleaning is necessary due to continuous use, a user may separate the nozzle 300 from the accommodation container 600, and the guide container 500 coupled to the joint 700 may be separated to allow separation between the accommodation container 600 and the guide container 500. Accordingly, after washing the nozzle 300, the accommodation container 600, and the piston 200, which are components covered with the foam-type material, the nozzle 300, the accommodation container 600, and the piston 200 may be reassembled and recycled in a clean state.

Next, the configuration for pressurizing the foam-type material contained in the accommodation container 600 to be discharged to the outside through the nozzle 300 will be described. Especially, the button 100, the piston 200, the pad member 400, and the guide container 500 will be described.

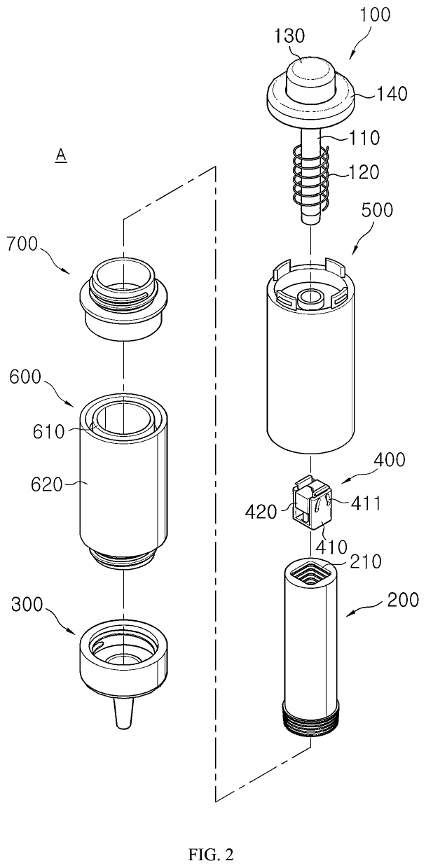

First, the guide container 500 is formed in a cylinder shape with the piston 200 inside, as shown in FIGS. 2 to 4, and is provided with the button 100 at the top thereof, and the joint 700 coupled to the accommodation container 600 may be coupled to the bottom of the guide container 500. The space formed inside the guide container 500 is preferably formed to have a length shorter than the length of the piston 200, so that when the piston 200 provided in the space is located at the uppermost side of the space, the lower end of the piston 200 is located within the accommodation container 600. In addition, because the piston 200 moves along the spaces formed inside the guide container 500 and the accommodation container 600 when the button 100 is pressed, the spaces formed inside the guide container 500 and the accommodation container 600 are preferably formed to have the same diameter.

The piston 200, which moves along the spaces formed inside the guide container 500 and the accommodation container 600, may be formed so that the remaining portion, excluding the lower end, has a diameter somewhat smaller than the diameter of each of the spaces formed inside the guide container 500 and the accommodation container 600. The lower end of the piston 200 is preferably formed to have a diameter somewhat larger than the diameter of each of the spaces formed inside the guide container 500 and the accommodation container 600, so that the piston 200 may be moved while being in close contact with the inner wall of the accommodation container 600. The lower end of the piston 200 may be formed in a concave-convex shape toward the inner wall of the accommodation container 600 so that the piston 200 may move when pressed without excessive friction.

In addition, the piston 200 may have a movement guide hole 210 recessed from the top downward, and the movement guide hole 210 may extend to near the lower end of the piston 200, and the pad member 400 that moves the piston 200 forward in conjunction with the button 100 may be provided in the movement guide hole 210.

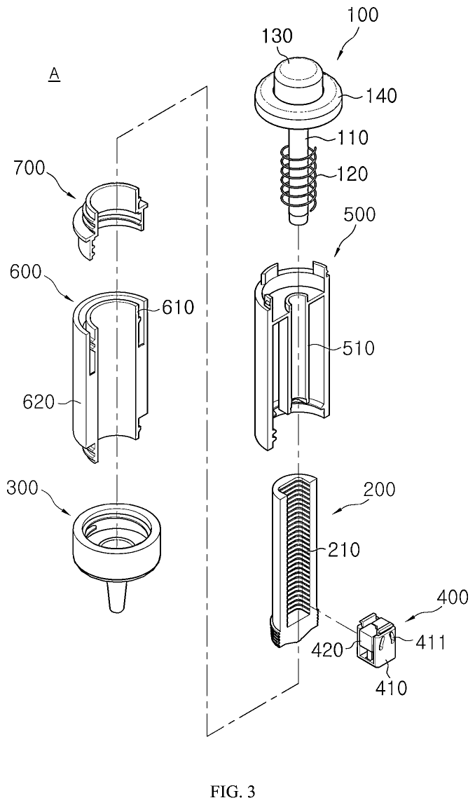

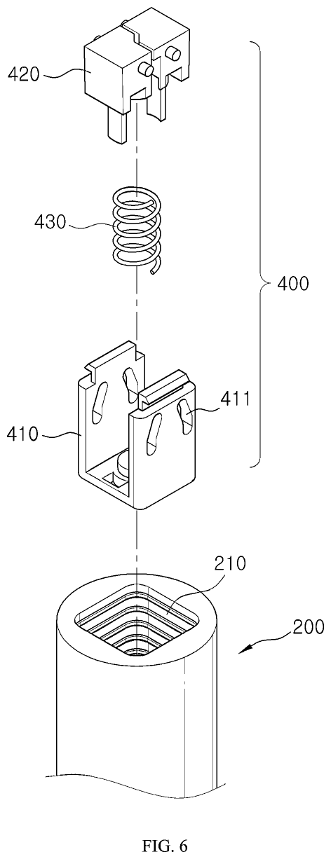

The pad member 400 is provided at a certain position within the movement guide hole 210 so as not to move even if the piston 200 is moved. The pad member 400 comes into contact with the wall of the movement guide hole 210 when the button 100 is pressed and advances the piston 200 a certain distance, and when the button 100 is released, the movement guide hole 210 is separated from the wall of the movement guide hole 210. As an example, as shown in FIGS. 3, 4, and 6, the pad member 400 may include: a pad support 410 fixed at a certain position in the movement guide hole 210; and a pad 420 that is movably coupled to the pad support 410 and moves downwardly outward when the button 100 is pressed, pushing the wall of the movement guide hole 210 to move the piston 200 a certain distance.

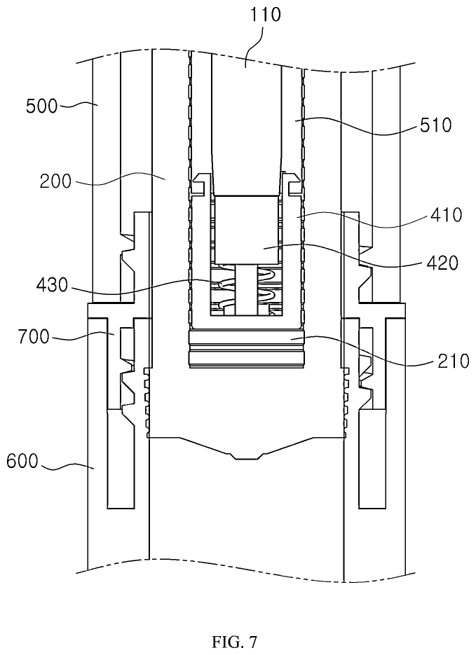

To be specific, the pad support 410 may be coupled to the lower end of a guide column 510 formed in the guide container 500 as shown in FIG. 7 so as to be fixed at a certain position within the movement guide hole 210. The guide column 510 has a rod shape with both ends open, and is formed downward from the center of the upper end of the guide container 500, and may be disposed in the movement guide hole 210. At this time, the lower end of the guide column 510 is preferably located at the lower part of the guide container 500. As the lower end of the guide column 510 and the upper end of the pad support 410 are coupled, the pad member 400 may be fixed at a certain position even if the piston 200 moves.

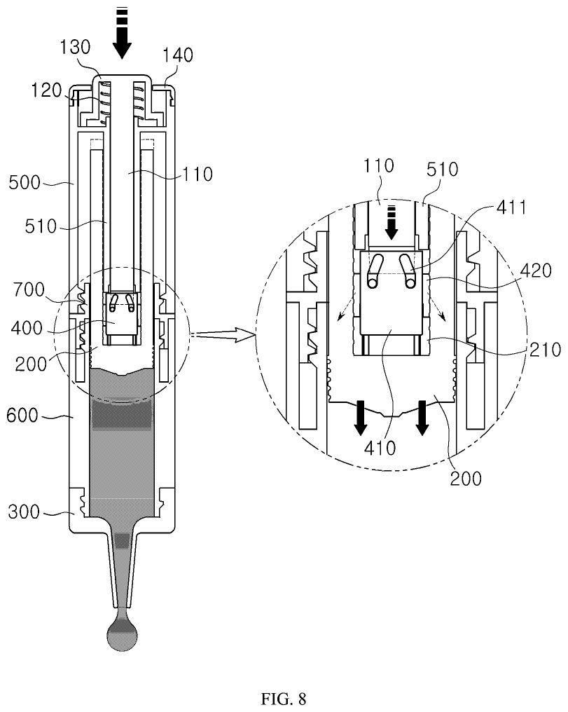

In addition, as an example, the pad support 410 may be arranged to be open upward, with side walls thereof formed upward so as to face each other on opposite sides of the bottom surface of the pad support 410, so that the longitudinal cross-section of the pad support 410 forms a “E” shape. A movement hole 411 may be formed in a diagonal direction on the side wall of the pad support 410 to provide a path so that the pad 420 may be coupled and moved downward toward the outside. As an example, the pad 420 may be pin-coupled to the movement hole 411 so that two pads 420 can be moved symmetrically, and when placed at the top of the movement hole 411, the pad 420 does not contact the wall of the movement guide hole 210. As shown in FIG. 8, when a downward force is applied by pressing the button 100, a pin of the pad 420 moves away from the top of the movement hole 411 so that the outer surface of the pad 420 comes into contact with the movement guide hole 210, and as the pin moves to the bottom of the movement hole 411, the outer surface of the pad 420 pushes the movement guide hole 210 in a downwardly inclined direction outward. As a result, the piston 200 moves downward, and the foam-type material contained inside the accommodation container 600 is discharged to the outside through the nozzle 300.

At this time, to enable the piston 200 to move easily according to the movement of the pad 420, wrinkles may be formed at regular intervals on the wall of the movement guide hole 210, and a locking protrusion corresponding to the wrinkles may be formed on the outer surface of the pad 420.

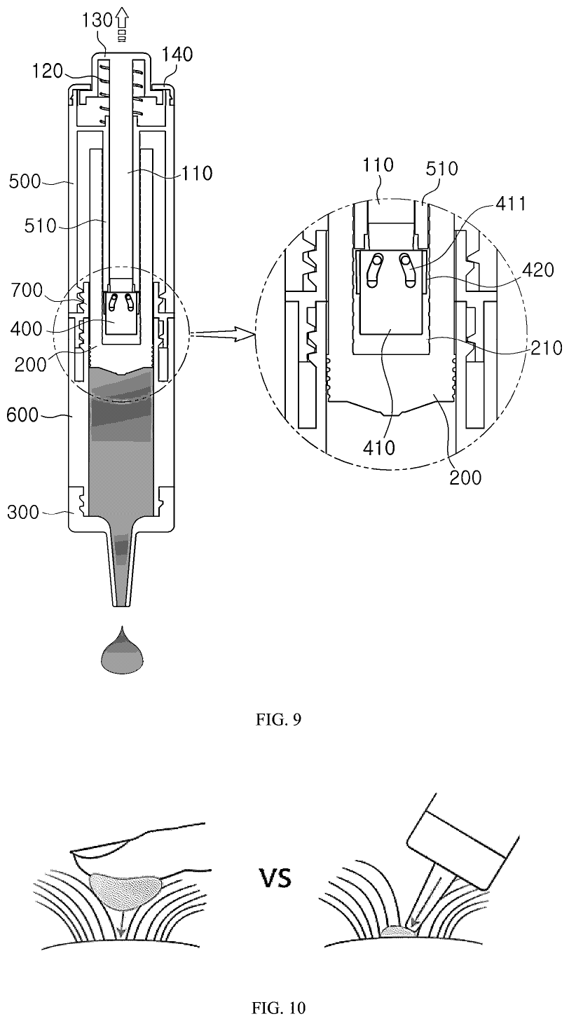

As shown in FIG. 9, when the button 100 is released, the pad 420 moves upward along the movement hole 411, and the pad member 400 may include a second spring 430 so that the pad 420 may be elastically returned when the button 100 is released. The second spring 430 has one end fixed to the bottom surface of the pad support 410 and the other end in contact with the bottom of the two pads 420, so that the second spring 430 is compressed when the button 100 is pressed, and then elastically expands when the button 100 is released, thereby moving the pad 420 upward.

The pad 420 operating as described above moves a certain distance along the movement hole 411 each time the button 100 is pressed and causes the piston 200 to advance a certain distance, so that a fixed amount of foam-type material may be ejected each time.

The button 100 may be configured to apply downward force to the pad 420 when pressed, and not apply force to the pad 420 when the button 100 is released. For this purpose, as shown in FIGS. 2 to 4, the button 100 may include: a pressing member 130 on which a user presses; a first spring 120 provided inside the pressing member 130 so that the pressing member 130 may be elastically returned to the original position thereof; a button cover coupled to the top of the guide container 500 to prevent the pressing member 130 from coming off; and a pressing column 110 that extends from the pressing member 130 and is formed to face the pad member 100 and is provided in the movement guide hole 210 to press the pad member 400.

Since the pressing column 110 is inserted into the guide column 510 located within the movement guide hole 210, the guide column 510 serves to provide a movement path for the pressure column 110. The pressing column 110 does not press the pad 420 coupled to the bottom of the guide column 510 when the pressing member 130 is not pressed, and presses the pad 420 when the pressing member 130 is pressed.

At this time, the pressing column 110 has one end in contact with the pad 420 formed in a wedge shape, and the two pads 420 that are pressed by the pressing column 110 and move symmetrically may have downwardly sloping surfaces on the upper surface thereof adjacent to each other so as to have an inclination corresponding to one end of the pressing column 110 formed in a wedge shape. Accordingly, when the pressing member 130 is pressed, the pressing column 110 has one end dug into the upper slopes of the pads 420, allowing the pads 420 to move smoothly outward and downward.

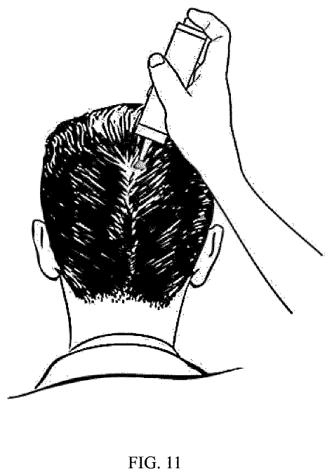

According to the foam-type material applicator of the present disclosure as described above, since a foam-type material may be discharged close to an application area such as the scalp, compared to applying with fingers as shown in FIG. 10, the temperature-sensitive foam-type material may be efficiently applied to the application area without phase change by not using fingers, and as shown in FIG. 11, a user may easily apply the foam-type material even to areas that are difficult to apply alone, such as the top of the head.

### Sequence Listing Free Text

### DESCRIPTION OF REFERENCE NUMERALS

A: applicator body
100: button 110: pressing column
120: first spring 130: pressing member
140: button cover
200: piston 210: movement guide hole
300: nozzle
400: pad member 410: pad support
411: movement hole 420: pad
430: second spring
500: guide container 510: guide column
600: accommodation container 610: inner container
620: outer container 630: filling space
700: joint

1. A foam-type material applicator, comprising:
an applicator body (A) in which a piston (200) provided thereinside advances by a certain distance when a button (100) is pressed so that a foam-type material accommodated thereinside is discharged outside through a nozzle (300),
wherein the applicator body (A) is configured such that the piston (200) remains in an advanced state when the button (100) is released so that a certain amount of the foam-type material is discharged whenever the button (100) is pressed.

2. The foam-type material applicator of claim 1, wherein the piston (200) is provided with a movement guide hole (210) recessed from a top thereof downward, and
the applicator body (A) further comprises a pad member (400) provided at a certain position in the movement guide hole (210), configured to contact a wall of the movement guide hole (210) and advance the piston (200) a certain distance when the button (100) is pressed, and spaced apart from the wall of the movement guide hole (210) when the button (100) is released.

3. The foam-type material applicator of claim 2, wherein the pad member (400) comprises:
a pad support (410) fixed at a certain position in the movement guide hole (210); and
a pad (420) movably coupled to the pad support (410), and configured to move downwardly outward when the button (100) is pressed and push the wall of the movement guide hole (210) to move the piston (200) a certain distance.

4. The foam-type material applicator of claim 3, wherein the button (100) comprises a pressing column (110) provided in the movement guide hole (210) to face the pad member (400) so as to press the pad member (400),
the applicator body (A) comprises a guide container (500) in which a guide column (510) is provided that surrounds the pressing column (110) within the movement guide hole (210) and provides a movement path for the pressing column (110), and in which the piston (200) is provided thereinside, and
the pad support (410) is fixed to a bottom of the guide column (510).

5. The foam-type material applicator of claim 1, wherein the applicator body (A) comprises an accommodation container (600) configured to accommodate the foam-type material thereinside and keep the foam-type material from changing phase, and
the nozzle (300) is detachably coupled to a bottom of the accommodation container (600).
